(19) United States
(12) Patent Application Publication
(10) Pub. No.: US 2026/0049974 A1
(43) Pub. Date: Feb. 19, 2026

(54) Method to define a production and/or application and/or use of a polymeric material

(71) Applicant: AT & S Austria Technologie & Systemtechnik AG, Leoben (AT)

(72) Inventor: Qi Tao, Leoben (AT)

(21) Appl. No.: 19/101,842

(22) PCT Filed: Aug. 31, 2022
(86) PCT No.: PCT/EP2022/074220

§ 371 Date: Feb. 6, 2025

(51) Int. Cl. G01N 33/44; B29C 37/00; B29K 105/00

(52) U.S. Cl. CPC G01N 33/442; B29C 2037/90; B29K 2105/0094

(57) ABSTRACT

The invention relates to a method to define a production and/or application and/or use of a polymeric material, comprising the steps of: estimation of a convention degree value (α) of the polymeric material based on at least one first measured value from the polymeric material relating to the kind of conversion degree and based on a fixed value (QI) associated with a respective reciprocal temperature (1/T) and a heating rate (β) of the specific polymeric material, the fixed value (QI) being independent from the conversion degree value (α) of the polymeric material, and definition of the production and/or the application and/or the use of the polymeric material use based on the estimated conversion degree value (α).

### FIELD OF THE INVENTION

The invention relates to a method to define a production and/or application and/or use of a polymeric material.

### BACKGROUND OF THE INVENTION

In general, a polymeric material may comprise one or more polymers, or a mixture of one or more polymers and one or more fillers. Polymeric materials, such as prepregs, solder masks, glues, ABFs etc., are important base materials for electronic devices and/or components of electronic devices, e.g., for PCBs, ECPs, and/or substrates. In addition, polymeric materials are widely used for coatings as paints, in fabrics for cloth etc. Further, the polymeric material may comprise one or more monomers and/or oligomers, wherein e.g. some glues and/or paints may consist to a high content of said structures. Understanding a behavior of these polymeric materials in terms of curing, ageing, and/or decomposition enables to define a production and/or application and/or use of the corresponding polymeric material. Therefore, curing kinetics of the polymeric materials are studied during a material qualification phase, e.g., in order to check whether the corresponding polymeric material is suitable for an intended production and/or application and/or use.

For studying the curing kinetics of the polymeric material, a Thermal Kinetic Analysis (TKA) may be carried out on a probe of the polymeric material, wherein the kinetic deals with measurement and parameterization of process rates and wherein the thermal analysis is concerned with thermally stimulated processes. TKA is widely used in all polymer-related industries for the curing and decomposition processes of the polymeric materials.

Further, Model Free Kinetics (MFK) is a well-known and widely used method for characterizing various polymeric materials, such as EMCs (Epoxy Moulding Compounds), Prepregs, SMs (Solder Masks), since it enables to predict the thermal behavior of the material, like the crystallization-, melting-, crosslinking- and decomposition-behavior, etc.

However, limited by the current MFK-theory, a prediction accuracy and a prediction range of the behavior of the polymeric material are highly influenced by the experimental data.

### DESCRIPTION OF THE INVENTION

It is an objective of the present invention to overcome at least some of the above-mentioned problems.

This objective is achieved by the subject-matter of the independent claim. Further exemplary embodiments are evident from the dependent claims and the following description.

An aspect of the invention relates to a method to define a production and/or application and/or use of a polymeric material, comprising the steps of: estimation of a conversion degree value of the polymeric material based on at least one first measured value from the polymeric material relating to the kind of conversion degree and based on a fixed value associated with a respective reciprocal temperature and a heating rate of the specific polymeric material, the fixed value being independent from the conversion degree value of the polymeric material, and definition of the production and/or the application and/or the use of the polymeric material use based on the estimated conversion degree value.

The polymeric material may comprise one or more polymers, or a mixture of one or more polymers and one or more fillers. Further, the polymeric material may comprise one or more monomers and/or oligomers, wherein e.g. some glues and/or paints may consist to a high content of said structures. The first measured value may be a first curing degree value at the given temperature, wherein the first measured value may be measured directly or may be determined from a measurement, e.g. from a TKA or DSC analysis, as explained later. While the first curing degree value may be determined by the measurement, the conversion degree value to be estimated may be any curing degree value at a desired temperature or time. This curing degree value can be estimated without another measurement. The fixed value may be referred to as Qi-Point. The Qi-Point may be determined in advance, as explained below.

The above method using the Qi-Point may be referred to as Model Free Kinetics with Qi-Point (MFKq). MFKq offers a guideline for a better control and processing of the polymeric materials. In addition, MFKq improves an accuracy of the conventional MFK method and enlarges the prediction range, in particular for non-isothermal and isothermal predictions. Meanwhile, it offers a way to judge a quality of the measurement data, in particular before making any prediction, in order to ensure a high quality of the prediction. Further, MFKq enables additional applications compared to conventional MFK, because of the enlarged prediction range. For example, MFKq may be used for a prediction of a state of not fully cured polymeric materials at certain environment temperature, e.g. for shelf lifetime prediction. The shelf lifetime prediction may be used for a guideline for the polymeric material storage time and/or temperature in a corresponding ware and/or storage house.

So, the advantages of the present invention relate to the use of the Qi-Point in the conversion degree estimation, allowing a more precise estimation of the values related to the specific conversion degree, e.g. the curing degree, at the respective temperature and heating rate. In other words, the estimation of the specific conversion degree, e.g. curing degree, of the polymeric material resulting from the specific temperature and the specific heating rate may be closer to the real conversion degree thanks to the adjustment of the estimated value through the involvement of the Qi-Point, as disclosed below, e.g. with respect to the preferred embodiments.

According to an embodiment, the definition of the production of the polymeric material comprises the step of adjustment of at least one parameter of the process for treating the polymeric material, the adjustment being based on the estimated conversion degree value. This may contribute to find a suitable parameter value for the process for treating the polymeric material.

According to an embodiment, the definition of the production of the polymeric material comprises the determination of the process time to reach a predetermined conversion degree value, the time determination being based on the estimated conversion degree value. This may contribute to program a device for converting the polymeric material.

According to an embodiment, the definition of the use of the polymeric material comprises the definition, in particular within a specific time slot, of the polymeric material use in function of the estimated conversion degree value. This may contribute to easily find the proper use case for the polymeric material.

According to an embodiment, the polymeric material is provided in a storage area, the use being defined in function of the estimated conversion degree value and the storage environment data. This may contribute to easily find the proper use case for the polymeric material.

According to an embodiment, the definition of the application of the polymeric material comprises the step of an application decision in a component in function of the estimated conversion degree value. This may contribute to easily make the proper application decision for the polymeric material.

According to an embodiment, the application decision is in function of the seat where the component is provided/applied. This may contribute to find the proper polymeric material for the corresponding seat.

According to an embodiment, the application decision in function of the seat where the component is provided/applied is in function of the surface and/or the material of the seat. This may contribute to find the proper polymeric material for the surface and/or the material of the corresponding seat.

According to an embodiment, the seat is at least a portion of a further component. This may contribute to find the proper polymeric material for the further component.

According to an embodiment, the method further comprises the step of: performing a Thermal Kinetic Analysis (TKA), specific to the conversion degree value under estimation, based on the first measured value, to estimate a slope of a Qi-curve, wherein the estimation of the conversion degree value of the polymeric material is based on the first measured value, the fixed value, and the estimated slope. This transformation of the values may be done to linearize the dependency of heating rate to the temperature. This may contribute to an easy graphical analysis of the adjusted estimated conversion degree value. If the first measured value, i.e. the first curing degree value, and the fixed value, i.e. the Qi-point, are connected in a Qi-curve diagram, the slope of the resulting line corresponds to the above slope. The Qi-curve diagram shows one or more Qi-curves. The Qi-curves each show the dependency of the natural logarithm of the heating rate versus the reciprocal temperature, wherein there is one Qi-curve per measured curing degree value.

According to an embodiment, the method further comprises the step of: adjustment of the estimated slope based on the fixed value; wherein the estimation of the conversion degree of the polymeric material is based on the measured value, the fixed value (the Qi-point), and the adjusted slope. This may contribute to an easy and reliable conversion degree estimation.

According to an embodiment, the acquisition of the first measured value from the polymeric material is performed for at least two values of the conversion degrees. This may allow the definition of the specific temperatures/heating rates and determining the Qi-Point in a very precise manner, for example through a graphical interception of the resulting estimated lines of the two specific estimated conversion degree values.

According to an embodiment, the at least two measured values comprise a minimum value and a maximum value of the specific conversion degree of the polymeric material. For example, in case of a curing rate as the conversion degree, the minimum value may refer to a polymeric material which is not cured and the maximum value may refer to a polymeric material which is fully cured. This may contribute to precisely defining the resulting estimated tendencies of the two specific estimated conversion degree values, and then the Qi-Point.

According to an embodiment, a third measured value is acquired from the polymeric material, the third measured value relating to the kind of conversion degree and for a further value of the conversion degree, the estimation of the conversion degree value of the polymeric material being based on the third measured value and the fixed value. This may contribute to a very accurate and easy estimation of the conversion degree value, for example through a graphical interception of the of the further conversion degree with the Qi Point.

According to an embodiment, the acquisition of the third measured value from the polymeric material relating to the kind of conversion degree and for a further value of the conversion degree is done independently from the acquisition of the measured values for the at least two values of the conversion degrees necessary to create a TKA. This may contribute to a very streamlined estimation of the conversion degree value, because once defined the two conversion degree tendencies and then defined the Qi Point, it is enough the measurement of one value (third measured value) to define the tendency of the further conversion degree graphically intersecting the value with the Qi Point.

According to an embodiment, the acquisition of one or more of the measured values of the polymeric material and/or the TKA are done in an isothermal and/or non-isothermal condition. This may contribute to an accurate deduction of the present MFKq theory.

According to an embodiment, the acquisition of one or more of the measured values of the polymeric material comprises measuring of the corresponding measured values at a specific position of the value change along the time and/or temperature. The specific position may be a peak, e.g. the maximum. A shift of the peak, e.g. the maximum, may be related to the corresponding thermodynamic theory. This may contribute to a very accurate acquisition of the one or more measured values of the polymeric material.

According to an embodiment, the kind of the conversion degree relates to a polymeric material degradation of the polymeric material and the respective TKA comprises a TGA (Thermogravimetric Analysis). The TGA may contribute to a very accurate TKA.

According to an embodiment, the kind of the conversion degree relates to a polymeric material viscosity (change) and the respective TKA comprises a rheological measurement. The rheological measurement may contribute to a very accurate TKA. The rheological measurement may be carried out with the help of a Rheometer.

According to an embodiment, the kind of the conversion degree relates to a curing rate change and the respective TKA comprises a DSC analysis. Alternatively, the respective TKA may comprise a DTA (Differential Thermal Analysis). The DSC analysis or the DTA may contribute to a very accurate TKA.

According to an embodiment, the kind of the conversion degree relates to a Modulus change and the respective TKA comprises a rheological measurement, e.g. a DMA measurement. The rheological measurement, in particular the DMA measurement, may contribute to a very accurate TKA.

According to an embodiment, the kind of the conversion degree relates to an uptake of one or more fluids and the respective TKA comprises a TMA (Thermomechanical Analysis)/humidity chamber and a TGA analysis-measurement. The fluids may be liquid, e.g. water, or gaseous, e.g. water vapor, ammonia, and/or CO₂.

According to an embodiment, the kind of the conversion degree relates to a CTE/shrinkage change and the respective TKA comprises a TMA measurement.

These and other aspects of the invention will be apparent from and elucidated with reference to the embodiments described hereinafter.

### BRIEF DESCRIPTION OF THE DRAWINGS

Below, embodiments of the present invention are described in more detail with reference to the attached drawings.

FIG. 1 shows a flowchart of an exemplary embodiment of a method to estimate a QI-point for a given polymeric material;

FIG. 2 shows an example of a DSC Thermogram of the polymeric material under different heating rates;

FIG. 3 shows an example of a diagram showing curing degrees of the polymeric material depending on the temperature at different heating rates under non-isothermal conditions;

FIG. 4 shows an example of an Ozawa-Flynn-Wall diagram;

FIG. 5 shows an example of a distribution of several intersections;

FIG. 6 shows a detailed view of intersections between 10% and 90% according to FIG. 5;

FIG. 7 shows examples of Qi-curves in a Qi-curve diagram;

FIG. 8 shows a detailed view of the QI-curves according to FIG. 7;

FIG. 9 shows a flowchart of an exemplary embodiment of a method for determining a graph for estimating a conversion degree value of a conversion degree of a polymeric material under isothermal conditions;

FIG. 10 shows a diagram including an exemplary graph of the conversion degree depending on the time under isothermal conditions;

FIG. 11 shows a flowchart of an exemplary embodiment of a method for determining a graph for estimating a conversion degree value of a conversion degree of a polymeric material under non-isothermal conditions;

FIG. 12 shows a diagram including an exemplary graph of conversion degree values depending on the temperature under non-isothermal conditions;

FIG. 13 shows a diagram including an exemplary graph of the conversion degree depending on the time under isothermal conditions which may be used for shelf-life time prediction;

FIG. 14 shows a flowchart of an exemplary embodiment of a method for determining a graph for estimating a conversion degree value of a conversion degree of a polymeric material under isothermal or non-isothermal conditions.

FIG. 15 shows a flowchart of an exemplary embodiment of a method to define a production and/or application and/or use of a polymeric material.

The reference symbols used in the drawings, and their meanings, are listed in summary form in the list of reference symbols. In principle, identical parts are provided with the same reference symbols in the figures.

### Detailed Description of Exemplary Embodiments

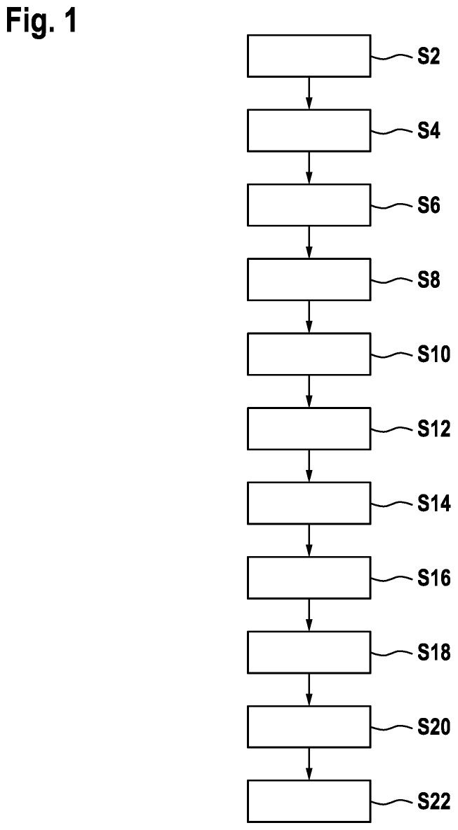

FIG. 1 shows a flowchart of an exemplary embodiment of a method to estimate a Qi-point QI (see FIG. 7) for a given polymeric material.

In a step S2, a Differential Scanning calorimetry (DSC) measurement of a probe of the polymeric material is carried out under n different heating rates βn°=°[β₀, β₁, . . . , βₙ₋₁], with n being a natural number. For example, n may be in the range from 1 to 20, e.g. from 1 to 10, e.g. from 3 to 6.

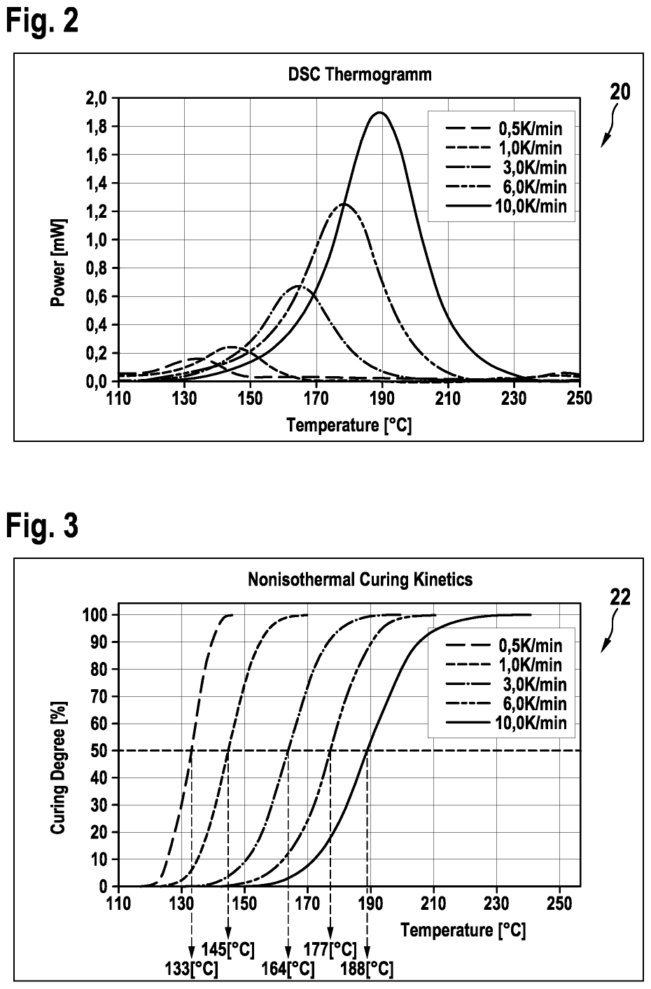

FIG. 2 shows an example of a DSC thermogram 20 of the polymeric material determined by the DSC measurement. The DSC thermogram 20 comprises one graph per heating rate β, wherein the heating rates βₙ are given in K/min. Alternatively, the heating rate β may be given in any other possible temperature to time relation, e.g. ° C./s or ° F./h.

A curing degree value a of a curing degree may be determined from the DSC thermogram 20, wherein the curing degree value a may be determined by the formula:

$$∝ = \frac{{H}_{T}}{{H}_{Total}} \times 100 \%$$

with H_{Total} being the energy which is absorbed by the polymeric material until the curing of the polymeric material is finished and with H_{Total} corresponding to the area under the corresponding graph of FIG. 2; and with H_{T} being the energy which is absorbed by the polymeric material until the temperature T is reached and with H_{T} corresponding to the area under the corresponding graph of FIG. 2 from the very left to the Temperature T.

In a step S4, the curing degree values α°=°[0, 0.01, . . . , 100] (in sum 10001 elements) and the corresponding temperatures Tᵢ=[T_{i,0}, T_{i,1}, . . . , T_{i,1000}] will be determined for each heating rate βᵢ, with i°=°0,°1, . . . , m−1 being a natural number.

FIG. 3 shows an example of a diagram, which may be referred to as first diagram 22 in the following. The first diagram 22 shows the determined curing degree values a of the polymeric material depending on the temperatures Tᵢ at the different heating rates Bn under non-isothermal conditions. From FIG. 3 it may be seen that the curing degree is a function of the heating rate B and the temperature T.

$$∝ = ∝ ( β , T )$$

For example, the dashed horizontal line within the first diagram 22 corresponds to a curing degree value a of 50%, i.e. α°=°50°%, wherein the intersections of the graphs of the curing degrees a with that dashed horizontal line provide the temperatures T at which the curing degree value a is 50% under the corresponding heating rate B.

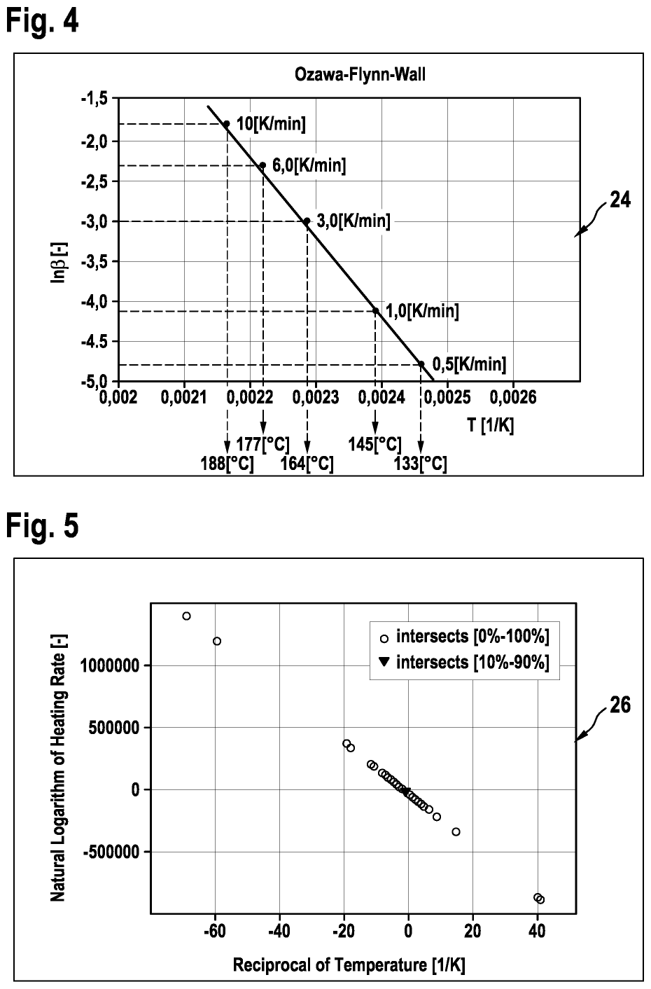

FIG. 4 shows an example of an Ozawa-Flynn-Wall diagram, which may be referred to as second diagram 24. The second diagram 24 may be achieved by an Ozawa-Flynn-Wall Analysis, as it is known in the art. The Ozawa-Flynn-Wall diagram of FIG. 4 may be constructed for the curing degree α°=°50% by the heating rates Bn and temperatures T extracted from the diagram of FIG. 3 by the corresponding horizontal line, as explained above.

The Ozawa-Flynn-Wall diagram may also comprise graphs correspondingly constructed for the other curing degrees α°=°[0, 0.01, . . . , 100]. However, the corresponding graphs constructed according to the conventional Ozawa-Flynn-Wall Analysis may intersect each other in a region of the Ozawa-Flynn-Wall diagram (not shown in the figures), what makes no sense from a physical point of view and what shows the drawbacks of the conventional Ozawa-Flynn-Wall Analysis. Therefore, instead of using the conventional Ozawa-Flynn-Wall Analysis, the present inventor found a more accurate way of constructing the graphs for predicting the curing degree a for the given polymeric material, as explained in the following.

In a step S6, the graphs in the Ozawa-Flynn-Wall diagram are plotted for all of the above Temperatures Tᵢ, heating rates Bn, and curing degrees α°=°[0,°0.01, ° . . . ,°100] according to

$$[ ( \frac{1}{{T}_{0 , j}} , ln {β}_{0} ) , ( \frac{1}{{T}_{1 , j}} , ln {β}_{1} ) , … , ( \frac{1}{{T}_{m - 1 , j}} , ln {β}_{n - 1} ) ]$$

with j°=°0, 1, . . . , 10000, for example. So, the corresponding diagram, which may be referred to as Qi-curve diagram, may e.g. comprise 10001 graphs.

In a step S8, the graphs of the Qi-curve diagram may be linearly fitted and the slopes and intercepts with the y-axis of the correspondingly fitted graphs, i.e. Qi-curves, may be extracted, resulting in slopes K°=°[k₀, k₁, . . . , k₁₀₀₀₀] and intercepts B°=°[b₀, b₁, . . . , b₁₀₀₀₀].

In a step S10, the intersections of the fitted graph for the curing degree value a between 1% and 30%, e.g. between 5% and 15%, e.g. α°=°10°% with all other fitted graphs may be determined, e.g. by

$$\{ \begin{matrix}ln β = {k}_{1000} \frac{1}{T} + {b}_{1000} \\ ln β = {k}_{i} \frac{1}{T} + {b}_{i} , ( i = 0 , … , 10000 , i \neq 1000 )\end{matrix}$$

$$[ ( \frac{1}{{T}_{0}} , ln {β}_{0} ) , ( \frac{1}{{T}_{1}} , ln {β}_{1} ) , … , ( \frac{1}{{T}_{9999}} , ln {β}_{9999} ) ]$$

In a step S12, the intersections of the fitted graph for the curing degree value a between 30% and 60%, e.g. between 40% and 55%, e.g. α°=°50°% with all other fitted graphs may be determined, e.g. by

$$\{ \begin{matrix}ln β = {k}_{5000} \frac{1}{T} + {b}_{5000} \\ ln β = {k}_{i} \frac{1}{T} + {b}_{i} , ( i = 0 , … , 10000 , i \neq 5000 )\end{matrix}$$

$$[ ( \frac{1}{{T}_{10000}} , ln {β}_{10000} ) , … , ( \frac{1}{{T}_{19999}} , ln {β}_{19999} ) ]$$

In a step S14, the intersections of the fitted graph for the curing degree value α between 70% and 99%, e.g. between 85% and 95%, e.g. α°=°90°% with all other fitted graphs may be determined, e.g. by

$$\{ \begin{matrix}ln β = {k}_{9000} \frac{1}{T} + {b}_{9000} \\ ln β = {k}_{i} \frac{1}{T} + {b}_{i} , ( i = 0 , … , 10000 , i \neq 9000 )\end{matrix}$$

$$[ ( \frac{1}{{T}_{20000}} , ln {β}_{20000} ) , … , ( \frac{1}{{T}_{29999}} , ln {β}_{29999} ) ]$$

In a step S16, the intersections determined in the steps S10 to S14 may be plotted in a third diagram 26, e.g. as shown in FIG. 5.

FIG. 5 shows an example of a distribution of several of the above intersections. The distribution of the intersections is shown in the third diagram 26. From FIG. 5 it may be seen that 80% of the intersections lie around the reciprocal temperature 0, wherein the reciprocal temperature of 0 may only be achieved for the temperature T going towards infinite.

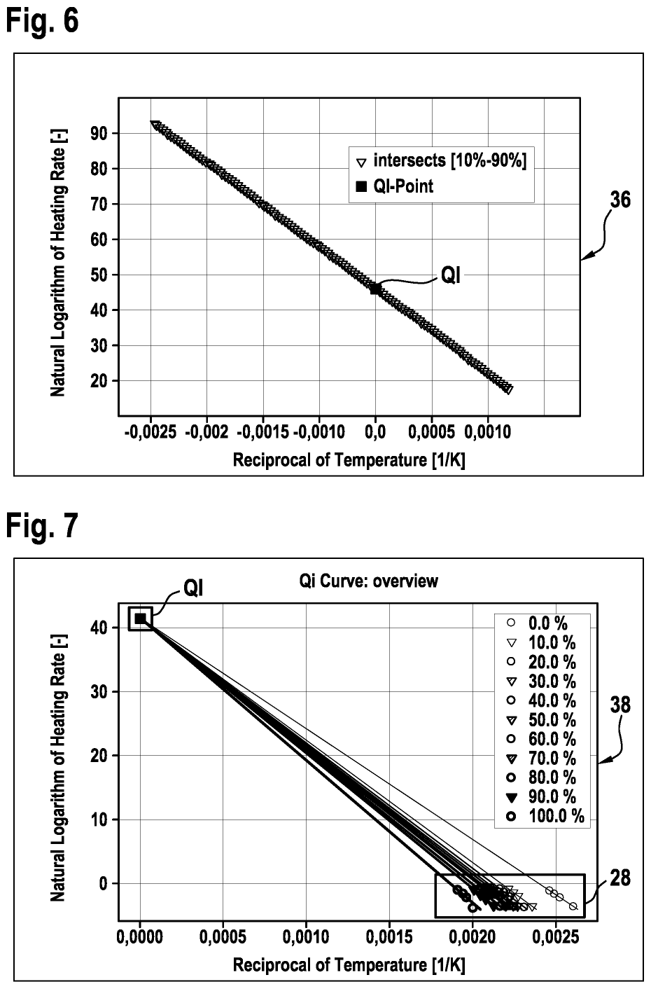

FIG. 6 shows a detailed view of the intersections between 10% and 90% in a fourth diagram 36.

In a step S18, the intersection of the fitted graph with 1/T°=°0 may be determined as the Qi-point QI, with QI°=°(0, In β).

FIG. 7 shows examples of Qi-curves, all of which including the Qi-point QI, in a fifth diagram 38. The Qi-curves do not intersect each other except for the Qi-point QI, what perfectly makes sense from a physical point of view.

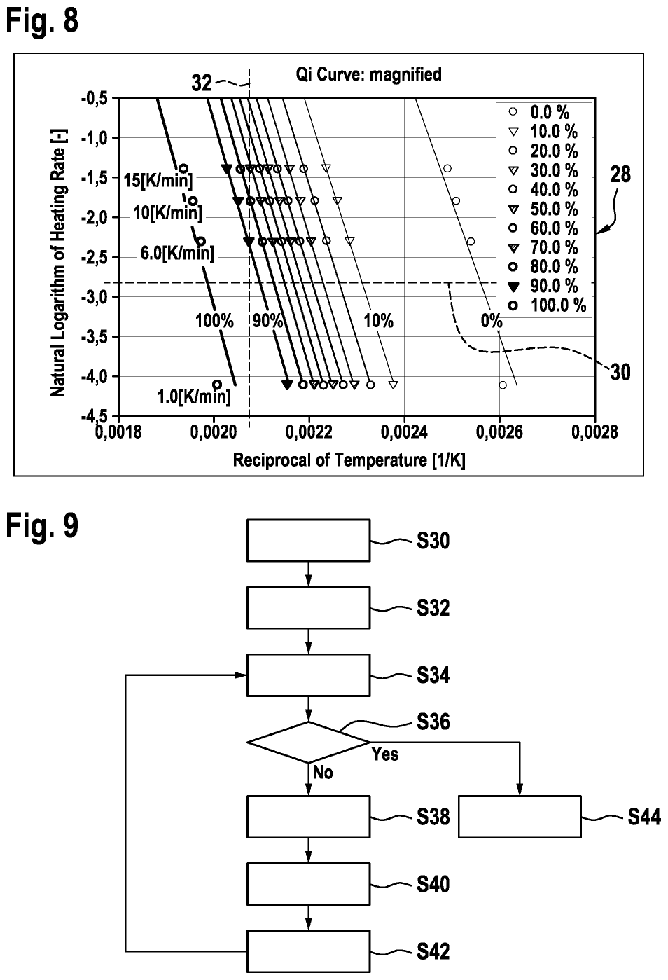

FIG. 8 shows a detailed view of the QI-curves according to FIG. 7, in particular a view of a lower area 28 of the fifth diagram 38.

The Qi-curves of FIGS. 7 and 8 may be determined by steps S20 and S22.

In the step S20, the graphs, i.e. the Qi-curves, are plotted again according to the Ozawa-Flynn-Wall equation

$$ln ( β ) = ( - \frac{{E}_{α}}{R} ) \frac{1}{T} + \{ ln [ Af ( α ) ] - ln ( \frac{d α}{d T} ) \}$$

and by using the Qi-point QI, wherein the term (−E_{α}/R) defines the slope, with R being the ideal gas constant, and the term {In [Af(α)]−In(d_{α}/dT)} defines the intercept of the corresponding curves, with A being the pre-exponential factor with the unit [1/s].

$$[ ( \frac{1}{{T}_{0 , j}} , ln {β}_{0} ) , ( \frac{1}{{T}_{1 , j}} , ln {β}_{1} ) , … , ( \frac{1}{{T}_{m - 1 , j}} , ln {β}_{n - 1} ) , ( 0 , ln {β}_{q} ) ]$$

with j°=°0, 1, . . . , 10000, for example.

In step S22, the graphs may be linearly fitted again in the Qi-diagram in order to obtain the Qi-curves, which correspond to the Ozawa-Flynn-Wall curves including the Qi-point QI, and correspondingly adapted slopes

K′=°[k′₀, ° k′₁, ° . . . ,° k′₁₀₀₀₀] and intercepts B′=°[b′₀, b′₁, . . . , b′₁₀₀₀₀] may be extracted.

FIG. 9 shows a flowchart of an exemplary embodiment of a method for determining a graph for estimating a conversion degree value a of a conversion degree of a polymeric material under isothermal conditions. The conversion degree may be the curing degree. Alternatively, the conversion degree is different from curing degree. In general, the conversion degree may describe a fraction of the reactant that already has reacted. The kind of conversion degree may depend on the type of the corresponding reaction. For example, if the reaction is a polymerization process, the conversion degree may be the curing degree. If the reaction is a decomposition process, the conversion degree may be a decomposition degree. Further, the conversion degree may be a polymer degradation, a polymer viscosity, a curing rate, a Modulus change, an uptake of one or more fluids, and/or to a CTE/shrinkage.

The method may use the above predetermined curing degree values ai, heating rates β and determined Qi-point QI and may determine the corresponding times t at which the predetermined curing degree values αᵢ are reached in order to estimate a continuous progression and/or behaviour of the curing degree values a under a given temperature Tiso in form of a mathematical function and/or a corresponding graph depending on the time such that one or more desired and/or arbitrary curing degree values a may be extracted by the mathematical function and/or from the corresponding graph afterwards.

In a step S30, the temperature Tiso is received for which the graph representing the curing degree values a of the curing degree of the polymeric material depending on the time t shall be estimated. The temperature Tiso may be input into a device for determining the graph for estimating the curing degree value a and the device may receive the temperature Tᵢₛₒ. The device may be a processor of a general purpose computer. The temperature Tᵢₛₒ may be received from an external device or from a memory of the general purpose computer.

In a step S32, the index i, the curing degree value αᵢ and the time tᵢ each are set to 0, and the above curing degree values α°=°[0, 0.01, . . . , 100] and the above Qi-point QI°=°(0, In β) are received by the device. Further, a slope of the graph may be given as a function of the curing degree a, e.g. by

$$f ( ∝ ) = \frac{ln ( \frac{100}{∝} - 1 )}{{c}_{3}} - {c}_{4}$$

wherein c₃ and c₄ may be determined by fitting the graph by a sigmoidal curve and wherein a has a value between predefined minimum value (amin) and maximum value (αₘₐₓ) different from αₘᵢₙ=0 and αₘₐₓ=100 respectively, for example αₘᵢₙ=2.5 and αₘₐₓ=97.5.

In a step S34, the index i is incremented by 1, i.e. i°=°j°+°1, and the next conversion degree value ai is chosen, as e.g. by αᵢ°=αᵢ₋₁°+°δα.

In a step S36, it is checked whether the current conversion degree value αᵢ is larger than 100. If the condition of step 36 is not fulfilled, the method may proceed in step S38. If the condition of step 36 is fulfilled, the method may proceed in step S44.

In step S38, ΔT may be determined by

$$Δ T = \frac{f ( {α}_{i} )}{ln ( {β}_{i} ) - ln ( {β}_{q} )} - \frac{f ( {α}_{i - 1} )}{ln ( {β}_{i} ) - ln ( {β}_{q} )} = [ \frac{f ( {α}_{i} )}{f ( {α}_{i - 1} )} - 1 ] {T}_{i s o}$$

In step S40, Δt may be determined by

$$Δ {t}_{i} = \frac{{\int }_{{T}_{i s o}}^{{T}_{i s o} + Δ T} {e}^{- \frac{{E}_{{α}_{i}}}{RT}} dT}{{β}_{i} ( {T}_{iso} , {α}_{i} ) {e}^{- \frac{{E}_{{α}_{i}}}{{RT}_{iso}}}} = \frac{{\int }_{{T}_{i s o}}^{{T}_{i s o} + Δ T} {e}^{\frac{f ( {α}_{i} )}{T}} dT}{{e}^{\frac{f ( {α}_{i} )}{{T}_{iso}} + ln ( {β}_{q} )} {e}^{\frac{f ( {α}_{i} )}{{T}_{iso}}}} = \frac{{\int }_{{T}_{i s o}}^{{T}_{i s o} + Δ T} EXP [ \frac{f ( {α}_{i} )}{T} ] dT}{EXP [ \frac{2 f ( {α}_{i} )}{{T}_{iso}} + ln ( {β}_{q} ) ]}$$

In step S42, tᵢ may be determined by tᵢ°=°tᵢ₋₁°+°Δt.

Then, the method proceeds in step S34.

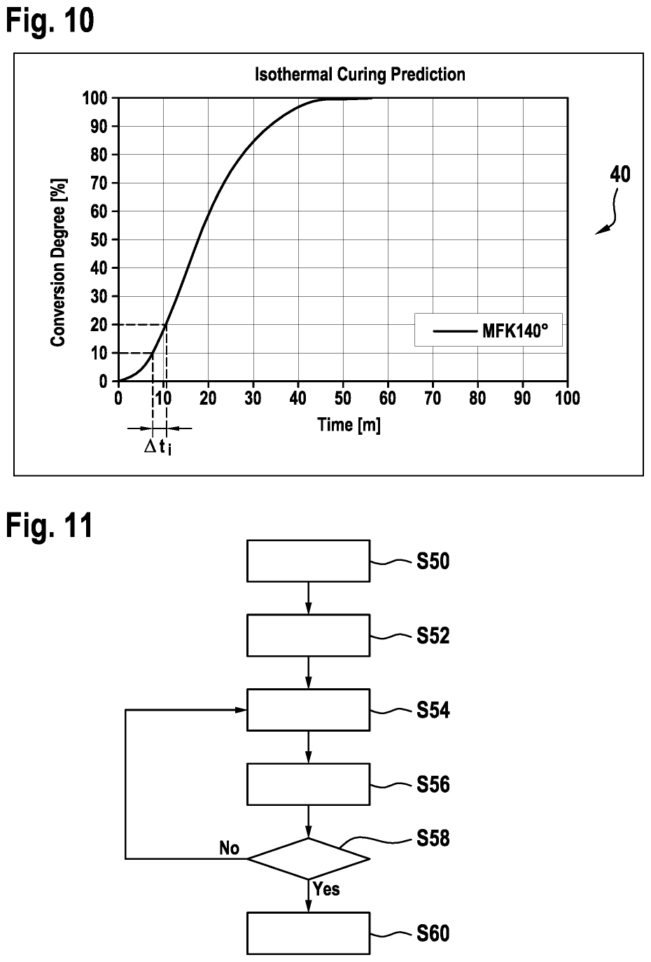

In step S44, the graph representing the behaviour of the curing degree values a over the time t may be plotted, e.g. as shown in FIG. 10, and/or the method for determining the graph for estimating the conversion degree values a of the conversion degree of the polymeric material under isothermal conditions may be terminated. This may have the advantage to predict curing times of polymeric materials more accurately.

The above method for determining the graph for estimating a conversion degree value a of the conversion degree of the polymeric material under isothermal conditions may be used as a sub-routine of a method for estimating the conversion degree value a of the conversion degree of the polymeric material under isothermal conditions. The latter method may estimate one or more conversion degree values a at a desired time t by extracting the corresponding conversion degree value a from the determined graph.

FIG. 10 shows a diagram including an exemplary graph of the conversion degree a depending on the time t under isothermal conditions. The diagram may be referred to as sixth diagram 40. The sixth graph 40 may be determined by the above method for determining the graph for estimating the conversion degree value a of the conversion degree of the polymeric material under isothermal conditions. The graph may be used by the method for estimating the conversion degree values a of the conversion degree of the polymeric material under isothermal conditions.

FIG. 11 shows a flowchart of an exemplary embodiment of a method for determining a graph for estimating a conversion degree value of a conversion degree of a polymeric material under non-isothermal conditions. The conversion degree may be the curing degree. The method may use the above predetermined curing degree values ai, heating rates β and determined Qi-point QI and may determine the corresponding temperatures T at which the predetermined curing degree values αᵢ are reached in order to estimate a continuous progression and/or behaviour of the curing degree values ai in form of a mathematical function and/or a corresponding graph depending on the temperature T such that one or more curing degree values a at one or more desired and/or arbitrary temperatures T may be extracted by the mathematical function and/or from the corresponding graph afterwards.

In a step S50, a given heating rate β is received for which the graph representing the curing degree values α of the curing degree of the polymeric material depending on the temperature T shall be estimated. The heating rate B may be input into the device for determining the graph for estimating the curing degree value a and the device may receive the heating rate B.

In a step S52, the index i and the curing degree value ai each are set to 0, and the above curing degree values αᵢ°=°[0, 0.01, . . . , 100] and the above Qi-point QI°=°(0, In β) are received by the device. Further, the slope of the graph may be given as the above function f(α).

In a step S54, Tᵢ may be determined by

$${T}_{i} = \frac{f ( {α}_{i} )}{ln ( β ) - ln ( {β}_{q} )}$$

In a step S56, the index i is incremented by 1, i.e. i°=°i°+°1, and the next conversion degree value αᵢ is chosen, as e.g. by αᵢ°=αᵢ₋₁°+°δα, wherein da may for example be 1.

In a step S58, it is checked whether the current conversion degree value ai is larger than 100. If the condition of step S58 is not fulfilled, the method may proceed in step S54. If the condition of step S58 is fulfilled, the method may proceed in step S60.

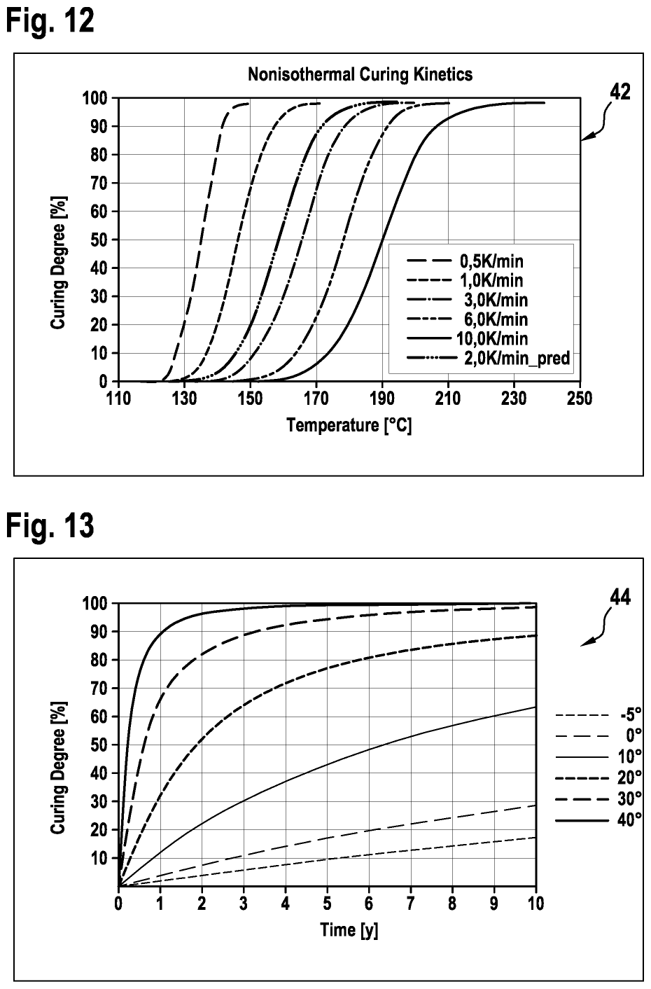

In step S60, the graph representing the curing degree a over the temperature T may be plotted, e.g. as shown in FIG. 12, and/or the method for determining the graph for estimating the conversion degree values a of the conversion degree of the polymeric material under non-isothermal conditions may be terminated.

The above method for determining the graph for estimating a conversion degree value a of the conversion degree of the polymeric material under non-isothermal conditions may be used as a sub-routine of a method for estimating the conversion degree value a of the conversion degree of the polymeric material under non-isothermal conditions. The latter method may estimate one or more conversion degree values a at correspondingly one or more desired temperatures T by extracting the corresponding conversion degree values a from the determined graph.

FIG. 12 shows a diagram including an exemplary graph of the conversion degree a depending on the temperature T under non-isothermal conditions, in particular for different given heating rates B. The diagram may be referred to as seventh diagram 42. The seventh diagram 42 may be determined by the above method for determining the graph for estimating the conversion degree value a of the conversion degree of the polymeric material under non-isothermal conditions. The graph may be used by the method for estimating the conversion degree values a of the conversion degree of the polymeric material under non-isothermal conditions.

Because of using the Qi-Point QI for fitting the Q-curves properly, they do not cross each other except for the Qi-Point QI. This enables to predict a shelf-life time of the corresponding polymeric material. For example, for predicting the shelf-life time of the polymeric material, it may be assumed that the corresponding time is very long, e.g. several years, and that the temperature stays constant during that time, as it is usual in corresponding storage depots.

FIG. 13 shows a diagram including an exemplary graph of the conversion degree depending on the time under isothermal conditions which may be used for shelf-life time prediction. The diagram shows the conversion degree depending on the time for different temperatures T. The diagram may be referred to as eighth diagram 44. The eighth diagram 44 may be determined by the above method for determining the graph for estimating the conversion degree value a of the conversion degree of the polymeric material under isothermal conditions. The time scale refers to years and reflects that some polymeric materials may be stored for years, in particular at about the same temperature, before they are used in a product.

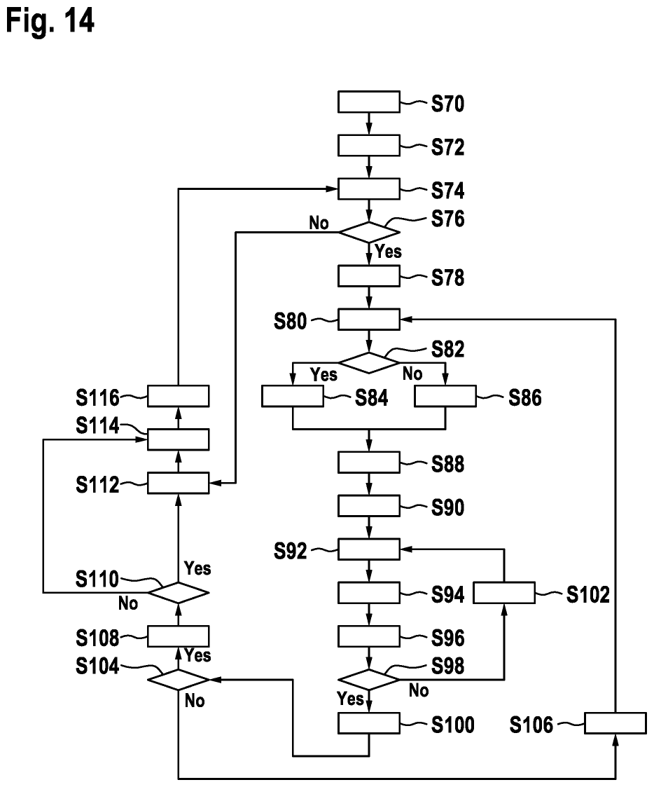

FIG. 14 shows a flowchart of an exemplary embodiment of a method for determining a graph for estimating a conversion degree value of a conversion degree of a polymeric material under isothermal or non-isothermal conditions. The conversion degree may be the curing degree. The method may use the above predetermined curing degree values ai, heating rates B and determined Qi-point QI and may determine the corresponding times t and/or temperatures T at which the predetermined curing degree values a; are reached in order to estimate a continuous progression and/or behaviour of the curing degree values a at a given time t and/or temperature T in form of a mathematical function and/or a corresponding graph depending on the time t and/or temperature T such that one or more curing degree values a at desired and/or arbitrary one or more times t and/or temperatures T may be extracted by the mathematical function and/or from the corresponding graph afterwards.

In a step S70, the predetermined temperatures Tᵢ=[T₀, T₁, . . . , Tₙ] and times t_{i°}=°[t₀, T₁, . . . , Tₙ] may be received by the device carrying out the method. Further, the curing degree value α is set to 0, δT is set to 0.1, and δα is set to 0.001, wherein the index i°=°0,°1,° . . . , °m−1 is a natural number.

In a step S72, the index i is set to 1 and the above Qi-point QI and slope f(α) are received by the device.

In a step S74, the curing degree value a is set to α=a₋₁.

In a step S76, it is checked whether the current conversion degree value ai is smaller than 100. If the condition of step S76 is not fulfilled, the method may proceed in step S112. If the condition of step S76 is fulfilled, the method may proceed in step S78.

In step S78, the temperature Tₛ is set to Tᵢ₋₁, e.g. by Tₛ°=°Tᵢ₋₁, with s being a natural number.

In a step S80, Δt₁ is set to 0.

In a step S82, it is checked whether

$$\vert {T}_{i} - {T}_{e} \vert < δ T$$

If the condition of step S82 is fulfilled, the method may proceed in a step S84. If the condition of step S82 is not fulfilled, the method may proceed in a step S86.

In step S84, Tₑ is set to Tᵢ.

In step S86, Tₑ is set in accordance with

$${T}_{e} = {T}_{s} + sign ( {T}_{i} - {T}_{i - 1} ) δ T$$

wherein the sign ( )-function may extract the sign of the real number within the brackets. This may enable to deal with not only heating applications but also with cooling down applications.

In a step S88, Δt is set in accordance with

$$Δ t = \frac{( {T}_{e} - {T}_{s} ) ( {t}_{i} - {t}_{i - 1} )}{{T}_{i} - {T}_{i - 1}}$$

In a step S90, Tᵢₛₒ is set in accordance with

$${T}_{i s o} = \frac{1}{2} ( {T}_{s} + {T}_{e} )$$

In a step S92, ΔT is set in accordance with

$$Δ T = [ \frac{f ( α + δ α )}{f ( α )} - 1 ] {T}_{i s o}$$

In a step S94, Δt₀ is set in accordance with

$$Δ {t}_{i} = \frac{{\int }_{{T}_{i s o}}^{{T}_{i s o} + Δ T} EXP [ \frac{f ( α + δ α )}{T} ] d T}{EXP [ \frac{2 f ( α + δ α )}{{T}_{i s o}} + ln ( {β}_{q} ) ]}$$

In a step S96, Δt₁ is set in accordance with

$$Δ {t}_{1} = Δ {t}_{1} + Δ {t}_{0}$$

In a step S98, it is checked whether

$$Δ {t}_{1} \geq Δ t$$

If the condition of step S98 is fulfilled, the method may proceed in a step S100. If the condition of step S98 is not fulfilled, the method may proceed in a step S102.

In step S100, the curing degree value a is set in accordance with

$$∝ = ∝ + δα \cdot \frac{Δ t - ( Δ {t}_{1} - Δ {t}_{0} )}{{t}_{0}}$$

In step S102, the curing degree value a is set in accordance with

$$∝ = ∝ + δα$$

In a step S104, it is checked whether

$${T}_{e} = {T}_{i}$$

If the condition of step S104 is fulfilled, the method may proceed in a step S108. If the condition of step S104 is not fulfilled, the method may proceed in a step S106.

In step S106, the temperature Tₛ is set to Tₑ.

In step S108, the curing degree value αᵢ is set to α.

In a step S110, it is checked whether

$${∝}_{i} > 100$$

If the condition of step S110 is fulfilled, the method may proceed in a step S112. If the condition of step S110 is not fulfilled, the method may proceed in a step S114.

In step S112, the curing degree value ai is set to 100.

In step S114, the function α.append(αᵢ) may be used for the corresponding program code, wherein a may be an array and may start with one element, i.e., α=[0]. When the corresponding loop starts, a will evolve from 0 to 100, which means the array needs to append the αᵢ one by one.

In a step S116, the index i incremented by 1.

The preceding method enables to plot graphs for the curing degree values under isothermal and non-isothermal conditions. In particular, the MFKq theory presented in this application enables find the correct curing degree value a by a given time t and temperature T. If the input of the time t and the temperature T is a non-isothermal case, then the output of the curing degree value α is for the non-isothermal case. If the input of the time t and the temperature T is in isothermal relationship, then the output of the curing degree a may stand for the isothermal curing condition.

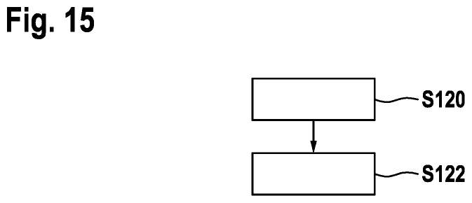

FIG. 15 shows a flowchart of an exemplary embodiment of a method to define a production and/or application and/or use of the polymeric material.

In a step S120, an estimation of the convention degree value a of the polymeric material is carried out based on the at least one first measured value from the polymeric material relating to the kind of conversion degree and based on the fixed value QI associated with the respective reciprocal temperature 1/T and the heating rate B of the specific polymeric material, with the fixed value QI being independent from the conversion degree value a of the polymeric material. The estimation may be carried out in accordance with one of the methods explained above, in particular depending on whether the corresponding measurements are carried out in an isothermal condition and/or a non-isothermal condition.

In as step S122, the production and/or the application and/or the use of the polymeric material is defined based on the estimated conversion degree value α. The definition of the production of the polymeric material may comprise a step of adjustment of at least one parameter of the process for treating the polymeric material, the adjustment being based on the estimated conversion degree value a. Alternatively or additionally, the definition of the production of the polymeric material may comprise a determination of a process time to reach a predetermined conversion degree value a, the time determination being based on the estimated conversion degree value a. Alternatively or additionally, the definition of the use of the polymeric material may comprise the definition, in particular within a specific time slot, of the polymeric material use in function of the estimated conversion degree value. In this context, the polymeric material may be provided in a storage area, the use being defined in function of the estimated conversion degree value a and the storage environment data. Alternatively, the definition of the application of the polymeric material may comprise a step of an application decision in a component in function of the estimated conversion degree value a. The application decision may be in function of the seat where the component is provided/applied. The application decision in function of the seat where the component is provided/applied may be in function of the surface and/or the material of the seat. In this context the seat is at least a portion of a further component.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

### LIST OF REFERENCE SYMBOLS

20 DSC Thermograph
22 first diagram
24 second diagram
26 third diagram
28 lower area
30 isothermal line
32 non-isothermal line
36 fourth diagram
38 fifth diagram
40 sixth diagram
42 seventh diagram
44 eighth diagram
QI Qi-point
S2-S122 steps two to one hundred twenty two

1. A method to define a production and/or application and/or use of a polymeric material, comprising the steps of:
estimation of a conversion degree value (a) of the polymeric material based on at least one first measured value from the polymeric material relating to a kind of conversion degree and based on a fixed value (QI) associated with a respective reciprocal temperature (1/T) and a heating rate (β) of the polymeric material, the fixed value (QI) being independent from the conversion degree value (α) of the polymeric material, and
definition of the production and/or the application and/or the use of the polymeric material use based on the estimated conversion degree value (a).

2. The method according to claim 1, wherein the definition of the production of the polymeric material comprises a step of adjustment of at least one parameter of a process for treating the polymeric material, the adjustment being based on the estimated conversion degree value (α).

3. The method according to claim 1, wherein the definition of the production of the polymeric material comprises a determination of a process time to reach a predetermined conversion degree value (α), the time determination being based on the estimated conversion degree value (α).

4. The method according to claim 1, wherein the definition of the use of the polymeric material comprises the definition, in particular within a specific time slot, of the polymeric material use in function of the estimated conversion degree value (a).

5. The method according to claim 4, wherein the polymeric material is provided in a storage area, the use being defined in function of the estimated conversion degree value (α) and a storage environment data.

6. The method according to claim 1, wherein the definition of the application of the polymeric material comprises the step of an application decision in a component in function of the estimated conversion degree value (α).

7. The method according to claim 6, wherein the application decision is in function of a seat where the component is provided/applied.

8. The method according to claim 7, wherein the application decision in function of the seat where the component is provided/applied is in function of a surface and/or the material of the seat.

9. The method according to claim 7, wherein the seat is at least a portion of a further component.

10. The method according to claim 1, further comprising the steps of:
performing a Thermal Kinetic Analysis (TKA) specific to the conversion degree value (α) under estimation, based on the first measured value, to estimate a slope of a Qi-curve, wherein the estimation of the conversion degree value (α) of the polymeric material is based on the first measured value, the fixed value (QI), and the estimated slope.

11. The method according to claim 10, further comprising the steps of:
adjustment of the estimated slope based on the fixed value (QI);
wherein the estimation of the conversion degree value (α) of the polymeric material is based on the first measured value, the fixed value (QI), and the adjusted slope.

12. The method according to claim 1, wherein an acquisition of the first measured value from the polymeric material is performed for at least two values of the conversion degrees.

13. The method according to claim 12, wherein the at least two measured values comprise a minimum value and a maximum value of the specific conversion degree of the polymeric material.

14. The method according to claim 12, wherein a third measured value is acquired from the polymeric material, the third measured value relating to the kind of conversion degree and for a further value of the conversion degree, the estimation of the conversion degree value (α) of the polymeric material being based on the third measured value and the fixed value (QI).

15. The method according to claim 14, wherein the acquisition of the third measured value from the polymeric material relating to the kind of conversion degree and for a further value of the conversion degree is done independently from the acquisition of the measured values for the at least two values of the conversion degrees necessary to create a TKA.

16. The method according to claim 15, wherein the acquisition of one or more of the measured values of the polymeric material and/or the TKA are done in an isothermal and/or non-isothermal condition.

17. The method according to claim 1, wherein an acquisition of one or more of the measured values of the polymeric material comprises measuring of the measured values at a specific position of a value change along time and/or temperature (T).

18. The method according to claim 15, wherein the kind of the conversion degree relates to a polymeric material degradation of the polymeric material and the TKA comprises a TGA analysis.

19. The method according to claim 15, wherein the kind of the conversion degree relates to a polymeric material viscosity change and the TKA comprises a Rheometer measurement.

20. The method according to claim 15, wherein the kind of the conversion degree relates to a curing rate change and the TKA comprises a DSC analysis.

21-23. (canceled)
